(19) United States
(12) Patent Application Publication
(10) Pub. No.: US 2005/0260555 A1
(43) Pub. Date: Nov. 24, 2005

(54) Chemosensitivity measurement using caspase activity

(75) Inventor: Franz Josef Meyer-Almes, Iserlohn (DE)

Correspondence Address: JACOBSON HOLMAN PLLC, 400 SEVENTH STREET N.W., SUITE 600, WASHINGTON, DC, 20004, US

(73) Assignee: Evotec Analytical Systems GmbH

(21) Appl. No.: 10/999,158

(22) Filed: Nov. 30, 2004

Related U.S. Application Data
(63) Continuation of application No. 09/762,304, filed on Apr. 16, 2001, now abandoned.

(30) Foreign Application Priority Data
Mar. 12, 1999 (DE) 199 10 956.7
Apr. 30, 1999 (EP) 99 108 495.5

(51) Int. Cl. C12Q 1/00

(52) U.S. Cl. 435/4

(57) ABSTRACT

A method for determining the chemosensitivity of cells towards at least one substance by measuring the apoptosis induced by said at least one substance, wherein the apoptosis is determined from the accumulated caspase activity of a sample comprising cells and a medium by adding said at least one substance to the sample followed by incubation, and measuring the accumulated caspase activity in the sample upon disruption of the cells without previously separating off the cells.

The present invention relates to a method for determining the chemosensitivity of cells towards at least one substance by measuring the apoptosis induced by said at least one substance.

Chemosensitivity Tests

Successful treatments and healings of tumor diseases have greatly increased in number since the introduction of chemotherapy. For example, the survival rate of infantile acute lymphatic leukemia (ALL) was less than 10% in the middle of the 1960s. Today, the chance of healing is above 70%. The medicaments, so-called cytostatic agents, are administered according to particular therapeutic plans alone or in combination with other active substances. By now, there are an immeasurable number of different therapeutic plans which have been established empirically and are constantly being developed further. The main criterion for an improved therapeutic plan is an improved survival rate (clinical outcome). This criterion is met by the majority of the patients. However, each individual has a different furnishing of cells with different properties. This is true, in particular, of the properties of tumor cells. In some studies, it could be shown that a therapy has an extremely good effect for one subpopulation while it is little effective or not effective at all for other patients because of drug-resistant tumors (Lacombe et al., Blood, 84, 716-723 (1994), Smit et al., International Journal of Cancer 51, 72-78 (1992)). It could be shown that not only the effectiveness of different medicaments but also the effective dose of one medicament can be different individually. Establishing the individual dosage of a medicament is important to patients in order to receive, on the one hand, as much medicaments as necessary for being healed and to keep, on the other hand, the toxic effects of the therapy and the probability of a secondary cancer disease induced by the chemotherapy as low as possible. Good as the currently usual therapeutic plans may be, progress in terms of average survival rate has been stagnating in recent years. A further significant improvement of chemotherapy should be possible by establishing individually matched therapeutic plans.

To approach these problems, some researchers have attempted to measure the individual sensitivity of patients' tumors towards cytostatic agents in vitro. Most chemosensitivity tests currently used are at least partially based on the agar tumor culture test developed by Salmon (Salmon et al., Science, 197, 461-463 (1977)). Such tests measure the proliferation of the cells.

A second type of chemosensitivity test comprises the exclusion of (fluorescent) dyes or the release of the radioactive chromium isotope ⁵¹Cr which measures the disruption of the cell membrane by the direct or indirect action of cytostatic agents. A modification of the earlier dye exclusion test is the so-called DiSC assay (Weisenthal, Kern, Oncology 5: 92-103 (1991)) which additionally differentiates between normal and tumor cells by a second dyeing process.

The third type of chemosensitivity tests determines parameters of cellular metabolism as a measure of damage done by cytostatic agents. This test type comprises the radiometric BACTEC test (von Hoff D., Forseth B., Warfel L., in Salmon Trent (Ed.), Human tumor cloning, pp., 656-657, Grune & Stratton, Orlando (1984)), the MTT test (Freund A. et al., European Journal of Cancer 34: 895-901 (1998), Kaspers G. J. L., Blood 92: 259-266 (1998), Pieters R. et al., Leukemia 12, 1344-1348 (1998), Klumper E. et al., British Journal of Haematology 93: 903-910 (1996), Hwang W. S. et al., Leukemia Research 17: 685-688 (1993)) and its variations, the ATP test (Kangas L., Gronroos M., Nieminen A., Medical Biology 62: 338-343 (1984)) and the so-called FCA test (Meitner P., Oncology 5: 75-81 (1991), Rotman B., Teplitz C., Dickinson K., Cozzolino J., Cellular and Developmental Biology 24: f1137-1146 (1988)).

Agar culture assays have a great disadvantage in that not all tumor cells by far will grow in the agar cultures. This is true, in particular, of lymphatic leukemias and lymphomas (Veerman A. J. P., Pieters R., British Journal of Haematology 74: 381-384 (1990)). In the best known representative of this assay type, the clonogenic assay, the percentage of cell populations which can be evaluated is as low as 30-40%. The cells have to grow for a very long time (10-20 days) before the evaluation is done. In addition, the expenditure of work is immense.

The dye exclusion tests and the ⁵¹Cr release test determine the proportion of cells having a defect cell membrane. These tests often include the fixation and subsequent microscopical evaluation of the cells or the measurement of the radioactivity in the supernatant. Since a rather rough parameter, the integrity of the cell membrane, is measured, such tests are non-specific and cannot distinguish between a specific anti-tumor activity of a substance and physical cell damage or cell damage caused by a substance, for example, by heavy erosion or oxidation. Consequently, test of this type give a positive result also with substances which generally cause damage to all cells and thus are not suitable as an anti-tumor medicament. The probably most telling test of the dye exclusion type is the DiSC test. In contrast to other dye exclusion tests, it is capable of differentiating between cell damage to tumor cells and to normal cells due to double staining. However, the double staining and subsequent microscopical evaluation are extremely time-consuming. In addition, the results vary as a function of the person performing the evaluation and the detail image observed with the microscope.

To radiometric tests, such as the ⁵¹Cr release test, the following applies quite generally: The use of radioactive isotopes in diagnostic tests is increasingly being avoided today because of the potential danger and the problems of disposal. In addition, by now, fluorescence and luminescence techniques can achieve the same sensitivities for a total duration of the test which is often even shorter.

Of the chemosensitivity tests making use of the metabolism of a cell as a measure of proliferation, the MTT test and its variations are used the most often. After 4 days of incubation of the cells with various cytostatic agents, the MTT test has a relatively short test duration of about 4 hours. In addition, 96 samples can be evaluated in parallel in a microtitration plate using an ELISA reading device. This ensures an acceptable throughput with a relatively low personnel expenditure. The MTT test is based on the conversion of 3-[4,5-dimethylthiazole-2-yl]-2,5-diphenyltetrazolium bromide into a blue formazan product. The conversion is catalyzed by dehydrogenases which are active only in living cells. The MTT test is dependent on a constitutive basic activity of the cells examined. However, it has been found that some cell types, e.g., particularly often some FAB subtypes of acute myeloic leukemia, have a highly reduced dehydrogenase activity (Santini V. et al., Hematological Oncology 7: 287-293 (1989)). Therefore, the chemosensitivity of these tumor cells cannot be evaluated with an MTT test. Another problem is the water-insoluble formazan crystals which form. They must be solubilized by the addition of organic solvents, such as dimethyl sulfoxide or isopropanol. Former experience has shown that this causes problems with the reproducibility of the tests.

Apoptosis

From the works of various research groups (e.g., Sen S. et al., FEBS Lett. 307: 122-127, Darzynkiewicz et al., Cytometry 13: 795-808 (1992), Fulda S., Los M., Friesen C., Debatin K. M., Int. J. Cancer 76: 105-114 (1998)), it appears likely that cell death caused by cytostatic agents generally involves the mechanism of apoptosis. Apoptosis is a cell death programmed by the cell itself which can be induced by physiological factors in the organism or by chemicals, such as cytostatic agents. The programmed cell death plays an extraordinarily important role in the cytostasis, e.g., of the immune system. Thus, for example, T cells directed against endogenous structures are removed from the organism by apoptosis. Otherwise, symptoms of an autoimmune disease may appear, e.g., lupus erythematodes, arthritic diseases, or disease from a T-cell tumor. Substances like the tumor necrosis factor alpha, which is synthesized by macrophages, are natural indicators of apoptosis.

Apoptosis proceeds according to a programmed uniform scheme in which particular cysteine-containing proteases (caspases) are activated (Cohen G. M., Biochem. J. 326: 1-16 (1997)). Caspases are activated exclusively by apoptosis and therefore are a specific parameter for the quantification of apoptosis. Up to the time of apoptosis, the cell membranes are still intact and impermeable to DNA-binding fluorescent dyes, such as propidium iodide. Therefore, caspase activity is a parameter for the activity of cytostatic agents which appears substantially earlier than the disruption of the cell membrane, which occurs hours later.

In addition to cell death by apoptosis, there is another form of cell death, that is necrosis, which is induced, e.g., physically, by osmotic shock, erosion or oxidation. This is manifested by a heavy degenerative damage to the cell.

Medenica (U.S. Pat. No. 5,736,129) determines the extent of cellular damage from cytostatic agents by staining the cells with propidium iodide, followed by counting the stained cells in a FACS (fluorescence-activated cell sorter). However, this approach has the great disadvantage of not precisely differentiating between apoptosis and necrosis; consequently, it cannot make use of the specific mechanism of action of cytostatic agents. In addition, the number of cells required for a telling analysis is very high.

Caspase activity tests are most suitable for the specific detection of apoptosis since necrotic damage, which is not relevant to cytostatic effects, is not covered (see Table 1). The methods used in recent years for measuring caspase activity comprise the gel-electrophoretic separation of specific protein substrates as well as chromogenic and fluorogenic tests in which colored products are formed by the caspases (see, e.g., Cohen G. M., Biochem. J. 326: 1-16 (1997), Stennicke H. R., Salvesen G. S., J. Biol. Chem. 41: 25719-25723 (1997)). All these tests are characterized in that the cells are first sedimented at a particular time after the induction and washed with a buffer, followed by disruption with a lysis buffer for the caspases, which had previously been present in the interior of the cell, to be able to convert the added substrate.

Thus, a catalogue of Clontech, Palo Alto, Calif., Catalogue 98/99, pages 71-72 (1998), describes a method for the determination of the apoptosis of cells in response to the action of substances. In this experimental design too, washing steps are performed according to the relevant protocols.

WO-A-99/18856 relates to a method for measuring the chemosensitivity of cells towards substances using cell-permeating fluorogenic substrates. There is a drawback in that only special substrates can be employed.

Fulda S. et al. disclose a method for measuring doxorubicin, cisplatin and VP-16, which induce apoptosis, using flow cytometry. Thus, cell lysates cannot be measured. In addition, flow cytometry is a comparably tedious method.

WO-A-98/13517 relates to a method for the estimation of the extent of apoptosis in cells. Thus, the cells are loaded with a substrate for proteases of the ICE/Ced-3 family, and the conversion of the substrate by the protease is followed by flow cytometry. Thus, however, cell lysates cannot be measured. As stated above, flow cytometry is relatively tedious. In addition, the loading of the cells requires that the cells be permeabilized. This may result in complications. Often, from the permeabilization treatment, the cells are no longer sufficiently authentic, but rather correspond to an artificial system.

However, the following problem arises from the washing procedure in view of a simple and quick performance of the test requiring little cellular material:

Apoptosis and thus the development of caspase activity is a dynamic process in which the cell proceeds to the so-called secondary necrosis after some time. Therefore, a washing procedure removes the caspases of these destroyed cells.

Thus, a signal for cells currently undergoing apoptosis is obtained, whose number will decrease sometime by secondary necrosis. It is known that the maximum of the number of apoptotic cells depends on both the cytostatic agent examined and the cell type. It is not known a priori at which time the caspase activity and thus the extent of apoptosis must be measured. It follows that, in principle, several samples with, e.g., tumor cells subjected to different incubation times must be assayed for chemosensitivity for every patient. The need of primary cells of patients would then be so high that the chemosensitivity towards only one or a few cytostatic agents could be measured. In addition, the time consumption and personnel expenditure for the chemosensitivity testing of a single patient would be extremely high.

It has been the object of the present invention to overcome the mentioned drawbacks of the prior art.

This object is achieved by a method for determining the chemosensitivity of cells towards at least one substance by measuring the apoptosis induced by said at least one substance, wherein the apoptosis is determined from the accumulated caspase activity of a sample comprising cells and a medium by adding said at least one substance to the sample followed by incubation, and measuring the accumulated caspase activity in the sample upon disruption of the cells without previously separating off the cells. In particular, the method according to the invention permits the use of substrates which are hardly cell-permeating or not cell-permeating at all.

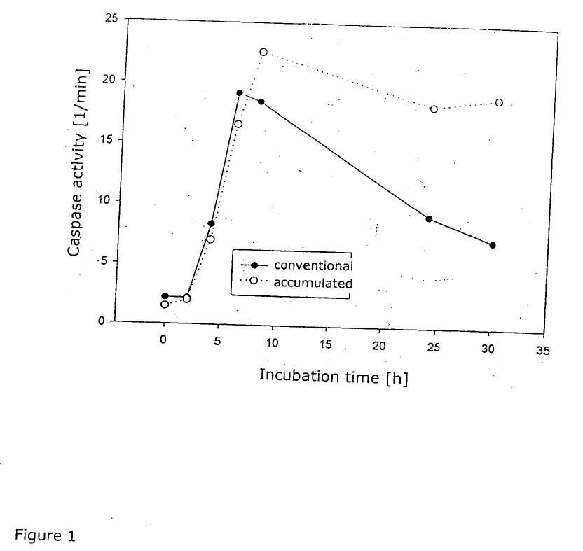

It is essential that the accumulated caspase activity is measured. In this approach, cells, e.g., tumor cells of a patient, are incubated with at least one substance (e.g., cytostatic agent) for an extended period of time so that all effective pharmaceuticals, even the slowly acting, have had enough time to induce apoptosis. Subsequently, for example, a substrate present in a lysis buffer is added, and after the cell lysis has been effected, the total caspase activity formed, i.e., the accumulated activity, of the cellular suspension is measured. It has been found that caspases activated by cytostatic agents remain active even after the secondary necrosis of apoptotic cells, and that the accumulated caspase activity increases with incubation time and remains on a high level from the time when secondary necrosis increasingly occurs. The conventionally determined (with a washing step) caspase activity increases at the same rate as the accumulated caspase activity, but decreases when the secondary necrosis begins (see FIG. 1). Thus, the principle of measuring the accumulated caspase activity permits to detect the effect of substances on the cells by a single caspase activity measurement. This test has a low need of cells and can be performed as a routine test by laboratory or technical assistants within a short time. Due to the easy automation, the personnel expenditure for performing the test is low. Since the test can be performed on ELISA reading devices, which are available everywhere, no investment in equipment is necessary. Miniaturization is possible to further reduce the consumption of materials. It could be shown that cells can be kept in culture for some days in less than 10 μl of medium. The reduction of the number of cells per sample compartment from 100 to 1000 per test enables highly parallel analyses of samples with extremely few cells from patients, e.g., from fine needle biopsies. The low need of cells also permits the high throughput screening (HTS) of unknown substances for anti-tumor activity.

When the chemosensitivity of cells from pathological tissues is tested using caspase activity, it is advantageous to include in the test healthy cells from the tissue as well as the same cells with no added substance as references. In particular, it is appropriate to include in the test as a control a permanent cell line whose chemosensitivity is known, in order to document the functionality of the test and realize mistakes in the test performance.

The following substances, for example, may be used as said substances:

A) Cytostatic agents

Abrin, amethopterin, acivin, aclacinomycin A, alanine mustard, altretarnine, aminoglutethimide, aminopterin, amsacrin (mAMSA), various anabolic steroids, anthrapyrazole, L-asparaginase, 5-axacytidine, Bacillus-calmette-guerin, bisantrene, busereline, busulfane, butyryloxyethylglyoxaldithiosemicarbazone, camptothecine, carbamate ester, carzinophyllin, CCNU, chlorambucil, chloroethylmethylcyclohexylnitrosourea, chloroethylcyclohexyinitrosourea,. chlorodeoxyadenosine, corticotropin, cyproterone acetate, chlorotrianisene, chlorozotozine, chromomycin A, cytosine arabinoside (Ara-C), BCNU, bleomycin, cisplatin, carboplatin, cladribine, cyclophosphamide, dactinomycin, daunomycin, daunorubicin, decarbacin, doxorubicin, DTIC, dehydroemitine, 4-demethoxydaunorubicin, demothydoxorubicin, deoxydoxorubicin, dexamethasone, dibromodulcitole, dichloromethotrexate, diethylstilbestrole, bis(2-chloropropyl)-DL-serine, doxifluridine, elliptinium acetate, 4′-epidoxorubicin, epirubicin, epoietinalpha, erythropoietin, esorubicin, estradiole, etoposide, fluoxymesterone, flutamide, folic acid, fotemustine, ftorafur, 4-FU, fludarabine phosphate, 5-FU, floxuridine, galactitole, gallium nitrate, gosereline, G-CSF, GM-CSF, hydrea, hexamethylmelamine (HMM), hydrocortisone, hydroxyprogesterone, 4-hydroperoxycyclophosphamide, ICRf 159, idamycin, ifosfamide, immunoglobulin IGIV, interferon, cobalt-proporphyrin complex, leucovorin calcium, leuprolide, leva-dopa, levothyroxine, lindane, liothyronine, liotrix, lomustine, levamisole, masoprocole, maytansine, menogaril, 6-mercaptopurine, methosalene, methylesterone, methyllomustine, mithracin, mithramycin, mitotane, mitoxanthrone, methotrexate (MTX), 6-MP, mechlorethamine hydrochloride, medroxyprogesterone, megestrole acetate, melphalane, mesna, mitomycin C, nandrolone, sodium phosphate P32, navelbine, neocarcinostatin, nitrofururazone, nHuIFNa, nHuIFNb, nHuIFNp, octreotide acetate, ondansetrone hydrochloride, disodium pamidronate, pentamethylmelamine (PMM), pentostatin, peptiochemio, plicamycin, prednimustine, probromane, procarbazine, profiromycin, paraplatin, prednisolone, prednisone, razoxane-rIFNa-2a, rubidazone, rIFNa-2b, rIFNb-1b, rIFNt-1b, rIL-2, rTNF, semustine, SPG 827 (podophylline derivative), spirogermanium, streptonigrine, somatostatin, streptozocin, tamoxifene, taxole, thio-TEPA, 6-thioguanine, tenoposide, testolactone, testosterone, 3-TGDR, rTNF, thyroglobulin, thyrotropin, trilostane, uracil mustard, VP-16, vincristine (VCR), vinblastine (VBL), verapamil, vindesine, vinzelidine, vitamin A acid, vitamin A analogues, zinostatin.

B) Peptides and peptoids.

C) Nucleic acids and nucleic acid derivatives.

D) Peptide nucleic acids (PNAs).

E) Hybrides of RNAS, DNAs, PNAs and derivatives.

The caspase activity can be measured both through the substrate turnover rate of fluorogenic or chromogenic substrates, or through the binding of specific markers, e.g., antibodies, F_{ab} fragments, single-chain antibodies, aptamers (structure-binding nucleic acids), and/or other proteins having binding sites for either unchanged (educts) or converted (products) caspase substrates. In particular, aminocoumarin-DEVD is suitable for use as a fluorogenic substrate.

Marker molecules which can specifically bind either educts or products of caspase reactions can comprise a dye portion, a colloidal metal (e.g., silver, gold), a radioactive isotope, and/or rare-earth metal chelates.

In particular, the accumulated caspase activity is measured no sooner than 10 h (especially from 24 to 48 h) after said at least-one substance has-been added to the sample, since only by then, even slowly acting cytostatic agents will have induced apoptosis. As apoptosis is a rather early indicator of the effectiveness of cytostatic agents, the test described is clearly quicker than for example, the MTT test with 4 days of incubation.

It is important that the caspase activities of equal cell numbers be compared upon apoptosis. Therefore, it is recommendable to standardize the extent of apoptosis for the total number of cells examined. This may be done, for example, by measuring the light absorption, scattered light, conductivity measurement, or microscopic counting. Standardization is necessary to be able to compare the extent of cellular damage caused by different cytostatic agents.

The method according to the invention is particularly suitable for the stratification of tumor diseases. According to the invention, a classification of the tumor diseases is enabled from which it can be seen which existing standard protocol for chemotherapy is appropriate. The test according to the invention permits to establish relatively quickly whether a particular tumor cell line will respond to a particular chemotherapeutic agent. Accordingly, it becomes possible to develop new protocols for the chemotherapy of tumor diseases by using the method according to the invention for the accumulated measurement of caspase activity as an indicator of the necrosis of tumor cells. Finally, the method according to the invention also allows to optimize an individual chemotherapy against tumor diseases by treating tumor tissue from a patient with chemotherapeutic agents and determining its response to these chemotherapeutic agents through apoptosis of the tumor cells according to the invention.

For example, a highly potent cytostatic agent which immediately stops the growth of cells and induces all cells to undergo apoptosis can exhibit significantly less apoptosis, due to the low number of cells, than is exhibited by a less potent cytostatic agent in the presence of which the cells can still proliferate at first and undergo apoptosis only later and to a low extent. Only standardization considers apoptosis in relation to the total number of cells and evaluates the more potent cytostatic agent correctly.

The chemosensitivity testing of cells can be performed to particular advantage with the aid of a kit. This kit comprises a sample support with several sample compartments, each sample compartment containing at least one substance. In addition, a standardized solution of a reagent for measuring caspase activity is also contained in each kit. In particular, the caspase substrate is aminocoumarin-DEVD in a lysis buffer. A caspase activity standard may also be contained in the kit. The sample support may be, for example, a commercially available microtitration plate. The selection of substances to be tested depends on the application; they may be provided ready to use in the compartments of the sample support either in solution or dried. The substances may be present either as dry substances, in solution, or in the presence of matrix substances, such as salts, buffers, carbohydrates, carboxylic acids, pyrimidines, inorganic or organic nanoparticles with diameters of up to 1 μm.

FIG. 1 shows the accumulated and conventionally measured caspase activities of Jurkat cells which were induced with actinomycin D for 4 h.

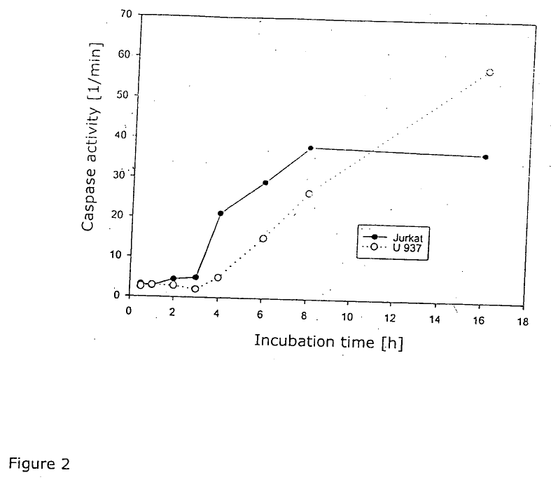

FIG. 2 shows the different time courses of apoptosis in Jurkat and U937 cells upon induction by actinomycin D.

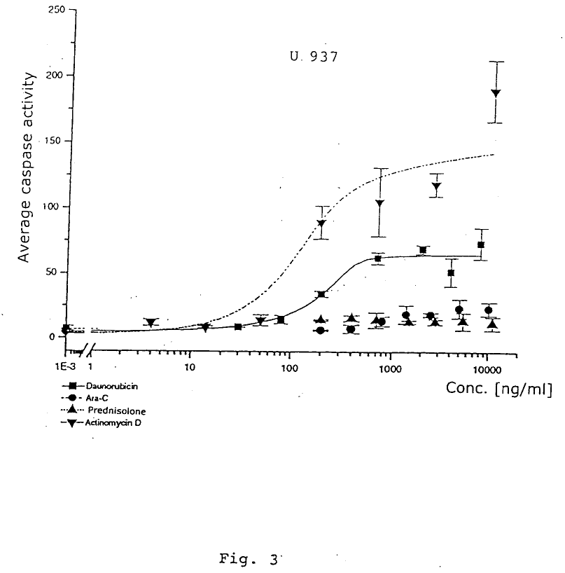

FIG. 3 shows the chemosensitivity of U937 cells towards different cytostatic agents.

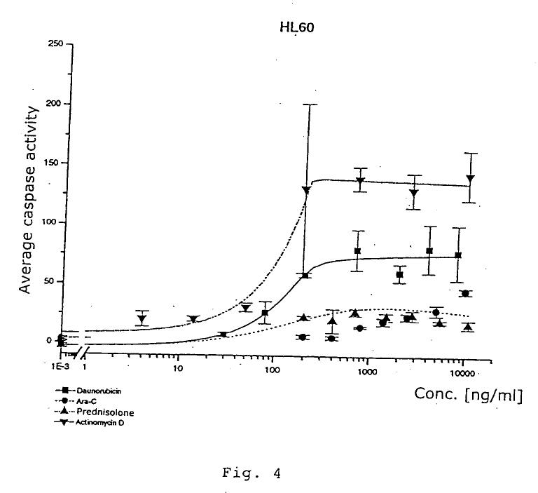

FIG. 4 shows the chemosensitivity of HL60 cells towards different cytostatic agents.

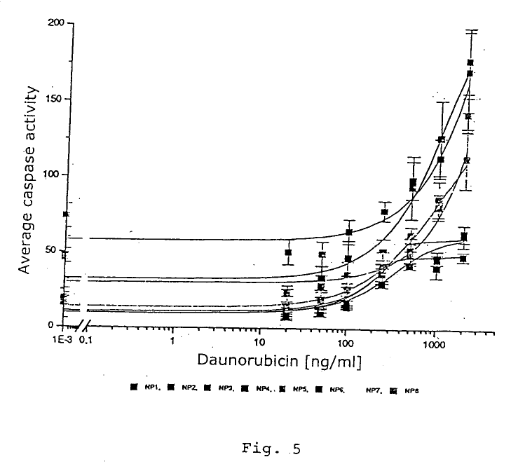

FIG. 5 shows the chemosensitivity of PBMNC towards daunorubicin.

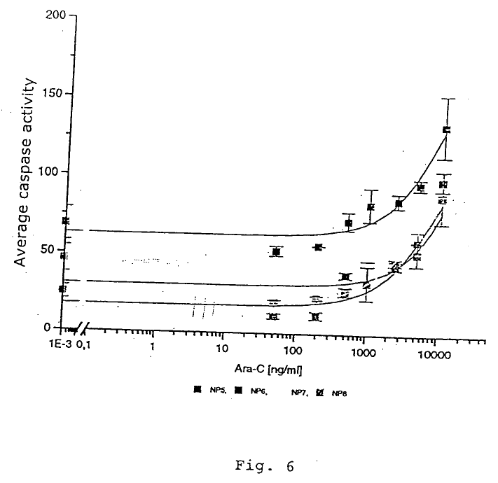

FIG. 6 shows the chemosensitivity of PBMNC towards Ara-C.

### EXAMPLES

### Example 1

The caspase activity of 250 000 Jurkat cells in 100 μl of medium was determined after different incubation times with 1 μg/ml actinomycin D both conventionally and according to the method of accumulated caspase activity according to the invention. Thus, 100 μl of Jurkat cells (2.5×10⁶/ml) was incubated in the presence of actinomycin D at 5% CO₂ and 37° C. in RPMI medium with 10% FCS and Pen/Strep. After the incubation, the cells were centrifuged by a conventional method and washed with PBS. The pellet was resuspended in 200 μl of lysis buffer, incubated on ice for 10 min and frozen at −20° C. The accumulated caspase activity was determined by mixing 100 μl of the cells incubated with actinomycin D, directly in suspension, with 100 μl of 2× lysis buffer. The lysate was also frozen at −20° C. All lysates were simultaneously thawed, and aminocoumarin-DEVD was added. At room temperature, the release of fluorescent aminocoumarin by caspase was monitored every 5 min over a period of 2 h. From the linear increase of the signal, the slope was calculated as a measure of caspase activity. The spontaneous caspase activities of the Jurkat cells in the absence of actinomycin D, not shown in the Figure, increase with time, but remain lower than 4 min⁻¹.

### Example 2

The caspase activities of Jurkat and U937 cells were determined after different incubation times with actinomycin D. Thus, 0.5×10⁶ cells were respectively incubated with 1 μg/ml actinomycin D in cell culture plates (48 wells) in 1 ml of RPMI 1640 medium with 10% FCS and Pen/Strep for between 30 minutes and 16 hours. Subsequently, the cells were transferred to Eppendorf cups and washed twice with medium. The pellet was then resuspended in lysis buffer. After 10 minutes on ice, 50 μM aminocoumarin-DEVD substrate was added, and the caspase activity was monitored at 37° C. every 5 min over a period of 2 hours. The fluorescence was excited at 355 nm, and the emission measured at 460 nm. The increase with time of the fluorescent emission is plotted against the incubation time with actinomycin D.

### Example 3

Chemosensitivity of Tumor Cell Lines

The chemosensitivities of U937 and HL60 cells towards the cytostatic agents indicated in FIGS. 3 and 4 were tested in the stated concentration ranges. It can be seen from the Figures that the two tumor cell lines exhibit differently strong responses to cytostatic agents, as would be expected in the case of primary cells from patients.

The effect of four cytostatic agents on the cell line U937 was tested. Each data point represents the average from three independent experiments. As their concentration increases, the effectiveness of actinomycin D, daunorubicin and Ara-C also increases, while prednisolone causes no or but a slight apoptosis induction.

The maximum inducible caspase activity is relatively low for Ara-C, while it is significantly higher for daunorubicin, and highest for actinomycin D.

For comparison, the chemosensitivity of promyeloic HL60 cells is shown in FIG. 2. In this case too, actinomycin D and daunorubicin induce higher caspase activities than Ara-C and prednisolone do. Prednisolone exhibits a maximum in caspase activity at about 700 ng/ml. At higher concentrations, the apoptosis signal decreases again. At the highest concentration measured, Ara-C appears not yet to have reached the maximum caspase activity inducible by this cytostatic agent.

### Example 4

Chemosensitivity of PBMNC

For establishing the test for peripheral blood or bone marrow, isolated cells from the peripheral blood (PBMNC) of healthy volunteers were tested first, taking care that acute infections, as frequently encountered in the fall/winter season, could be excluded. FIGS. 5 and 6 illustrate the results of the studies with two cytostatic agents.

FIG. 5 shows the effect of daunorubicin on the mononuclear cells of eight subjects (NP); in each case, the average±standard deviation from three independent experiments is shown. Caspase activity can be induced by >50 ng/ml daunorubicin; however, a plateau, i.e., an unchanging maximum caspase activity, is not reached for the subjects NP1 and NP5 to NP8, and only roughly so for the subjects NP2 to NP4. As compared to tested leukemia cell lines, the PBMNCs respond later to daunorubicin.

FIG. 6 shows the effect of Ara-C on the mononuclear cells of subjects NP5 to NP8; in each case, the average±standard deviation from three independent experiments is shown. Caspase activity can be induced by >200 ng/ml Ara-C; however, a plateau, i.e., an unchanging maximum caspase activity, is not reached for any of the tested PBMNCs.

As compared with the data from the cell lines HL60 and U937 (Example 3), it is evident that the maximum inducible caspase activity is lower for those two cell lines.

1-11. (canceled)

12. A method for determining the chemosensitivity of cells towards at least one substance, by measuring the apoptosis induced by the at least one substance, comprising:
a) incubating a sample comprising cells and a medium with at least one substance and
b) measuring the accumulated caspase activity in the sample upon disruption of the cells without previously separating cells and medium from each other, the accumulated caspase activity being measured utilizing a fluorogenic substrate comprising the sequence motif DEVD,
wherein the extent of apoptosis is commensurate with the chemosensitivity of the cells towards the substance.

13. The method according to claim 12, wherein the cells are animal cells, including human, cells.

14. The method according to claim 12, wherein the cells are human, cells.

15. The method according to claim 14, wherein the cells are leukemia cells, cells of solid tumors, cells of pathological organs, and/or reference cells.

16. The method according to claim 14, wherein the reference cells are cells from organs other than the pathological ones, or cells from healthy regions of pathological organs.

17. The method according to claim 12, wherein the substance is a pharmaceutically active substance.

18. The method according to claim 17, wherein the pharmaceutically active substance is a chemotherapeutic agent.

19. The method according to claim 12, wherein the substance is selected from the group consisting of environmental pollutants, peptides, nucleic acids, PNAs, nucleic acid hydrides, and derivatives thereof.

20. The method according to claim 12, wherein the caspase activity is measured through a turnover rate of the fluorogenic substrate.

21. The method according to claim 12, wherein the accumulated caspase activity is measured no sooner than 10 h after the at least one substance has been added to the sample.

22. The method according to claim 21, wherein no sooner than 10 h is 24 to 28 h.

23. The method according to claim 12, wherein the caspase activity measured is standardized for the total number of cells.

24. The method according to claim 12, wherein the fluorogenic substrate is part of a lysis buffer added to the sample for disruption of the cells.

25. The method according to claim 12, wherein the fluorogenic substrate includes a dye portion or a rare-earth chelate.

26. In a method for developing new chemotherapies of tumor diseases, the method comprising determining the chemosensitivity of tumor cells toward a substance, the improvement comprising determining the chemosensitivity by the method of claim 12.
